(19) United States
(12) Patent Application Publication
(10) Pub. No.: US 2009/0169480 A1
(43) Pub. Date: Jul. 2, 2009

(54) DENDRITIC POLYMERS AND MAGNETIC RESONANCE IMAGING CONTRAST AGENT EMPLOYING THE SAME

(75) Inventors: Dhakshanamurthy Thirumalai, Hsinchu City (TW); Chin-I Lin, Yongkang City (TW); Shian-Jy Jassy Wang, Hsinchu County (TW)

Correspondence Address: LOWE HAUPTMAN HAM & BERNER, LLP, 1700 DIAGONAL ROAD, SUITE 300, ALEXANDRIA, VA, 22314, US

(73) Assignee: INDUSTRIAL TECHNOLOGY RESEARCH INSTITUTE, HSINCHU (TW)

(21) Appl. No.: 12/174,431

(22) Filed: Jul. 16, 2008

(30) Foreign Application Priority Data
Dec. 31, 2007 (TW) 96151446

(51) Int. Cl. A61K 49/06

(52) U.S. Cl. 424/9.3

(57) ABSTRACT

A dendritic polymer and a magnetic resonance imaging contrast agent employing the same. The magnetic resonance contrast agent includes the dendritic polymer according to the structure of

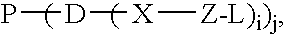

wherein, P is CH₂CH₂O₁, and 1 is not less than 1 and j is not less than 2; D is a C₃₋₃₀ dendritic moiety having n oxygen residue, and n is not less than 3 and i is more than 1; X is C₃₋₃₀ moiety having bi-functional groups; Z is independent and includes a C₃₋₂₀ moiety having a plurality of functional group, wherein the functional groups are selected from a group consisting of carbonyl, carboxyl, amine, ester, amide, or chelate group, and Z respectively bonds with X by a

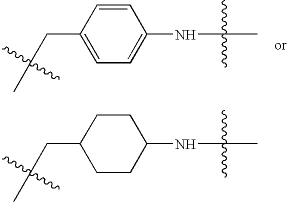

group; and L is a metal cation.

### CROSS REFERENCE TO RELATED APPLICATIONS

This application is based upon and claims the benefit of priority from the prior Taiwan Patent Application No. 96151446, filed on Dec. 31, 2007, the entire contents of which are incorporated herein by reference.

### BACKGROUND OF THE INVENTION

1. Field of the Invention

The invention relates to a magnetic resonance imaging contrast agent, and more particularly to a magnetic resonance imaging contrast agent containing dendritic polymers.

2. Description of the Related Art

Currently, medical imaging is capable of creating functional and anatomical image via physical signals of magnetic, photo (fluorescence, near-infrared, X-ray), and radioactive rays emitted by different imaging instruments. The imaging instruments include the Planar X-ray Imaging system, the X-ray Computerised Tomography (CT) system, and the Magnetic Resonance Imaging (MRI) system, which are utilized in the diagnosis of the central nervous system, skeletal nervous system, stomach, ribcage, angiography, diagnosis of biliary tractphotography, and the diagnosis of mutation of tumor tissue. The appearance of anatomic tissue does not change, but the change in blood circulation, cell activity, and metabolism of the location has been occurred in many clinical symptoms. Therefore, the location of the illness can be detected early by high sensitivity Nuclear Imaging.

Diethylenetriaminepentaacetic acid (DTPA) ligands are widely used in fundamental research as useful chelators in magnetic resonance imaging. These complexes also reduce longitudinal and transverse relaxation times (T1 and T2 respectively) of water molecule protons, resulting in a pronounced contrast enhancement in an magnetic resonance image.

However, clinically used contrast agent as Magnevist (gadolinium salt of DTPA) reveal a disadvantage. Immediately after intravenous application, it clears quickly from the body through the glomerulus of the kidney and leakage from the vessels as the low molecular weight compound. With this rapid clearance rate, physician has limited ability to complete time-dependent imaging studies or obtain highly resolved images of patients. Small molecular contrast agents are unable to detect anomalies smaller than a few centimeters using magnetic resonance imaging (MRI), thereby requiring high concentration of the contrast agent.

Nevertheless, not only does the risk of toxicity caused by highly concentrated heavy metal occur, but an abundance of molecular imaging agents also abundantly accumulate in the same portion. Thus, clinical applications are limited.

Accordingly, a desired magnetic resonance imaging agent is developed in order to efficiently target the location of the illness with lower dosage, which is a significant topic of research in magnetic resonance imaging technology. To overcome the disadvantages such as rapid clearance rate from the body and the need of high local concentration rendered by small molecular contrast agents, a high-molecular weight magnetic resonance imaging agent with multiple chelates is developed in order to efficiently target the location of the illness with lower dosage, which is a significant topic of research in magnetic resonance imaging technology.

### BRIEF SUMMARY OF THE INVENTION

The invention provides a dendritic polymer and a magnetic resonance imaging contrast agent, employing the dendritic polymers, capable of recognizing an affected part of a human body with high sensitivity. A detailed description is given in the following embodiments with reference to the accompanying drawings.

An embodiment of a dendritic polymer according to the structure of formula (I) is provided.

PDX-Z-L)ᵢ)ⱼ formula (I)

P is CH₂CH₂O₁, 1 is not less than 1, and j is not less than 2.

D is independent and comprises a C3-30 dendritic moiety having n oxygen residue, and n is not less than 3, and D respectively bonds with P and X by the oxygen residual groups.

X is a C3-30 moiety having bi-functional groups, such as

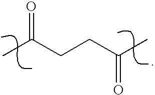

Z is independent and comprises a C3-20 moiety having a plurality of functional group and terminal

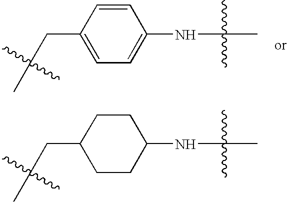

group, wherein the functional groups are selected from a group consisting of carbonyl, carboxyl, amine, ester, amide, or chelate group, and Z respectively bonds with X by

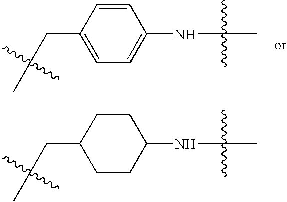

group. For example, Z is

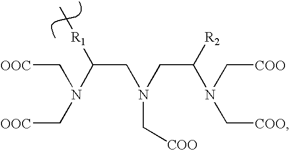

wherein R1 is

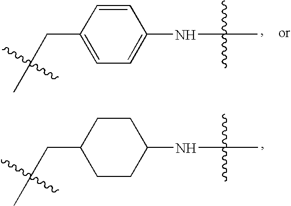

and R2 is H, methyl, ethyl, or propyl.

Further, Z comprises a residual group of ethylenedinitrilo tetraacetic acid (EDTA) or a residual group of ethylenediimino dibyric acid (EDBA) which with terminal

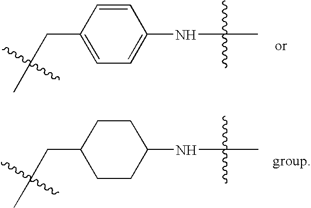

L is a metal cation or analyte-specific moiety; and i is not less than one and i can equals or less than n−1.

For the polymers contained in the magnetic resonance imaging contrast agent in the embodiments, P can be any conventional binding segment of ethylene glycol and its derivatives, preferably polymer segments of poly ethylene glycol. In some embodiments, D is a C3-30 dendritic moiety with n oxygen residual groups which can be bonded with D and a plurality of Z. Preferably, D is 2,2-dihydroxymethyl propanoic acid and residual groups of the derivative thereof, such as

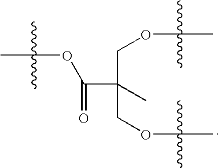

Besides, D can be a dendrimer moiety with layers of unrestricted numbers, preferably 2 to 3 layers, such as

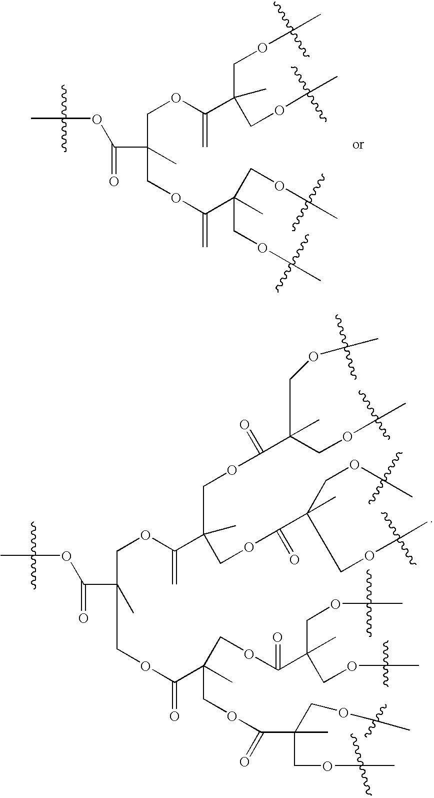

The metal cation in some embodiments is capable of taking part in physiological metabolism as a developing agent with high sensitivity and precision of magnetic resonance, such as Gd³⁺. According to some embodiments, the analyte-specific moiety is a molecular moiety specifically reacted with a specific target, such as a folic acid group, a glucose group, or an amino acid group. In some embodiments, Z can be a chelated agent, such as a residual group of ethylenedinitrilo tetraacetic acid (EDTA) or a residual group of ethylenediimino dibyric acid (EDBA). In addition, Z can be

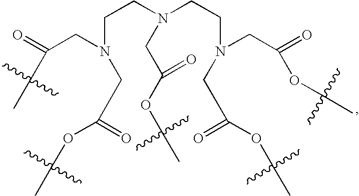

wherein Z bonds with D by one oxygen atom and bonds with L by the other oxygen atoms.

A detailed description is given in the following embodiments with reference to the accompanying drawings.

### DETAILED DESCRIPTION OF THE INVENTION

The following description is of the best-contemplated mode of carrying out the invention. This description is made for the purpose of illustrating the general principles of the invention and should not be taken in a limiting sense. The invention is an improved bonding pad and method for their fabrication. Although the invention is described with respect to a specific embodiment, the principles of the invention, as defined by the claims appended herein, can obviously be applied beyond the specifically described embodiments of the invention described herein. Moreover, in the description of the invention, certain details have been left out in order to not obscure the inventive aspects of the invention. The details left out are within the knowledge of a person of ordinary skill in the art.

### EXAMPLE 1

Preparation of dendritic polymer (A) containing metal cation Gd3+ with rigid linker

Preparation procedure of the polymer (A) is shown as below:

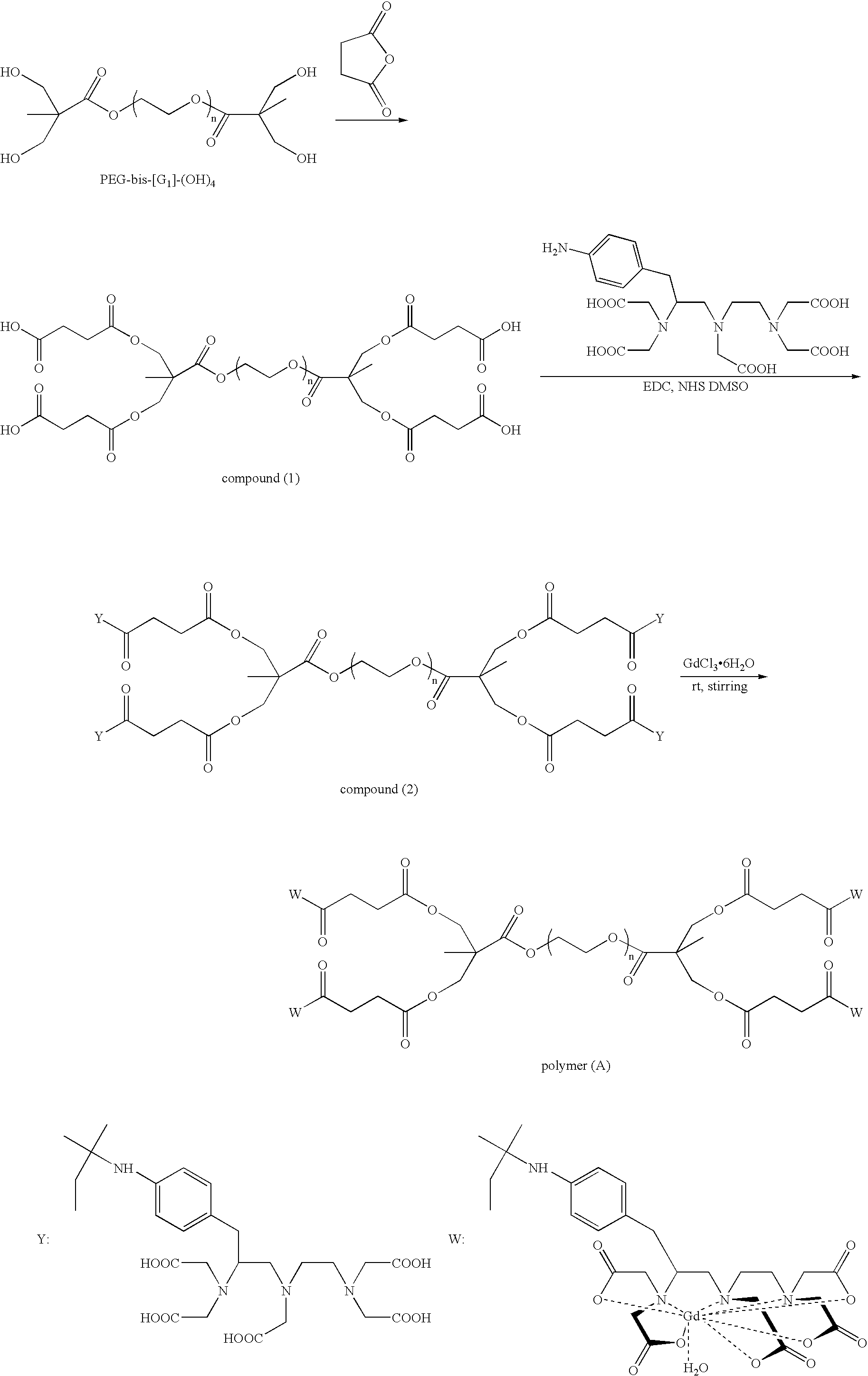

PEG-bis[G-2]-(OH)₄ (2.00 g, 0.43 mmol) and DMAP (0.020 g, 0.16 mmol) were dissolved in 75 mL of dichloromethane and succinic anhydride (0.37 g, 3.7 mmol) was added. The reaction mixture was stirred overnight and the mixture was precipitated in diethyl ether (2 L). The white precipitate separated was filtered and dried under vacuum, obtaining compound (1) (PEG-bis[G-1]-(OSA)₄) with a yield 91%. Physical measurement of the compound (1) is listed below:

¹HNMR (CDCl₃, 400 MHz): δ 1.20 (s, 6), 2.61 (s, 16), 3.68 (bs), 4.28 (t, 4).

A mixture of compound (1) (0.9 g, 0.19 mmol), EDC (0.12 g, 0.77 mmol), NHS (0.089 g, 0.77 mmol) and DMAP (0.095 g, 0.77 mmol) in DMSO (10 mL) was stirred at room temperature for 1-2 hr. Next, a solution of 2-(4-aminobenzyl)diethylenetriaminepentaacetic acid (0.387 g, 0.77 mmol) dissolved in 10 mL of DMSO was added dropwise with vigorous stirring. The stirring was continued for 48 hours and the reaction mixture was dialysed against DI water for about 3 days. Then the dialysed sample was lyophilized to afford the compound (2) (PEG-bis[G-1]-(SA-NH-Bz-DTPA)₄). with a yield 61%. Physical measurement of the compound (2) is listed below:

IR (cm⁻¹): 3395, 2811, 1732, 1643, 1108.

¹HNMR (CDCl₃, 400 MHz): δ 1.15, 1.19 (2s, 6), 2.44 (s, 16), 3.68 (bs), 4.13 (t, 4), 6.47 (d, 8), 6.84 (d, 8).

The polymer (A) containing metal cation Gd³⁺ with rigid linker was prepared by adding a stoichiometric amount of GdCl₃.6H₂O into compound (2) in water. The solution was vigorously stirred for 4 hr at room temperature. The pH maintained between 6.0-6.5 using 1N NaOH solution. The progress of the reaction was followed by FTIR. The absence of free gadolinium ions were tested by using xylenol orange indicator at pH 5.8 (acetate buffer). The complexes were filtered using 0.45 μm filter and lyophilized.

### EXAMPLE 2

Preparation of dendritic polymer (B) containing metal cation Gd³⁺ with rigid linker

Preparation procedure of the polymer (B) is shown as below:

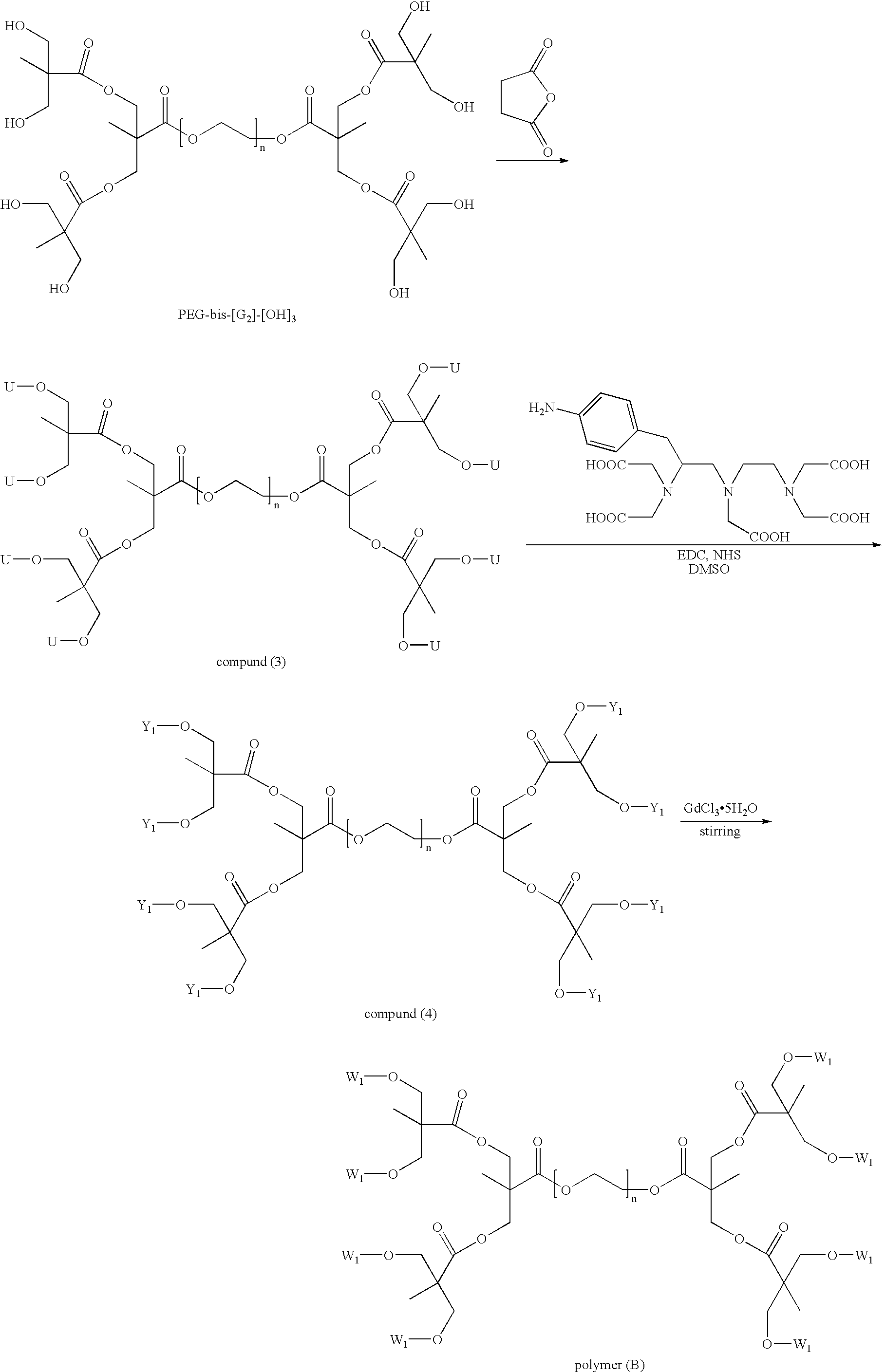

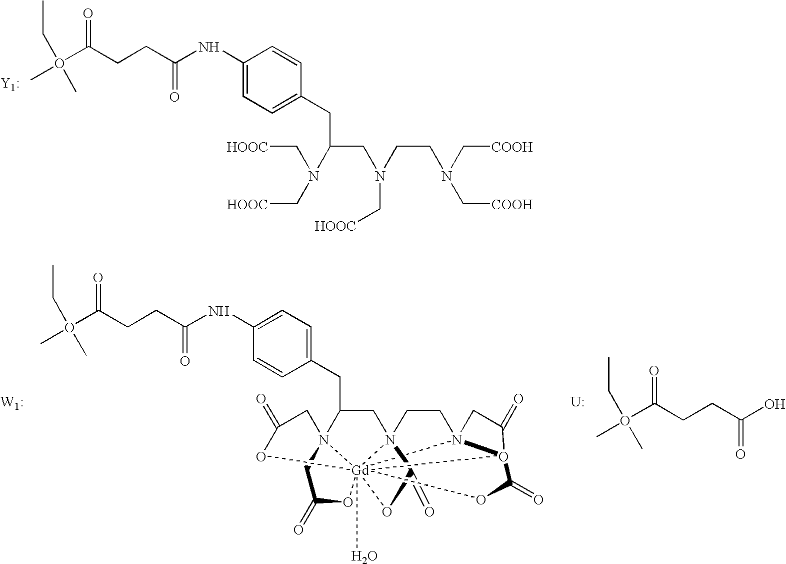

PEG-bis[G-2]-(OH)₈ (2.00 g, 0.43 mmol) and DMAP (0.020 g, 0.16 mmol) were dissolved in 75 mL of dichloromethane and succinic anhydride (0.37 g, 3.7 mmol) was added. The reaction mixture was stirred overnight and the mixture was precipitated in diethyl ether (2 L). The white precipitate separated was filtered and dried under vacuum, obtaining compound (3) (PEG-bis[G-2]-(OSA)₈). Physical measurement of the compound (3) is listed below:

¹HNMR (CDCl₃, 400 MHz): δ 1.15 (s,12), 1.20 (s, 6), 2.58 (2s, 32), 3.44 (m, 8), 3.61 (bs), 3.80 (t), 4.20 (m).

By following the general procedure, from a mixture of compound (3) (0.2 g, 0.036 mmol), EDC (0.054 g, 0.35 mmmol), NHS (0.0366 g, 0.32 mmol) and DMAP (0.039 g, 0.32 mmol) in DMSO (10 mL) and a solution of 2-(4-aminobenzyl)diethylenetriaminepentaacetic acid (0.173 g, 0.35 mmol) in 10 mL of DMSO, the compound (4) (PEG-bis[G-2]-(SA-NH-Bz-DTPA)₈) with a yield 55% was synthesized. Physical measurement of the compound (4) is listed below:

IR (cm⁻¹): 3416, 2874, 1738, 1644, 1110. ₁H NMR (400 MHz, DMSO-d₆): δ 0.95 (s, 12), 1.13 (s, 6), 2.44 (2s, 32), 3.05 (m, 8), 3.49 (bs), 4.16 (m), 6.99 (d), 7.56 (d).

The polymer (B) containing metal cation Gd³⁺ with rigid linker was prepared by adding a stoichiometric amount of GdCl₃.6H₂O into compound (4) in water. The solution was vigorously stirred for 4 hr at room temperature. The pH maintained between 6.0-6.5 using 1N NaOH solution. The progress of the reaction was followed by FTIR. The absence of free gadolinium ions were tested by using xylenol orange indicator at pH 5.8 (acetate buffer). The complexes were filtered using 0.45 μm filter and lyophilized.

### EXAMPLE 3

Preparation of dendritic polymer (C) containing metal cation Gd³⁺ with rigid linker

Preparation procedure of the polymer (C) is shown as below:

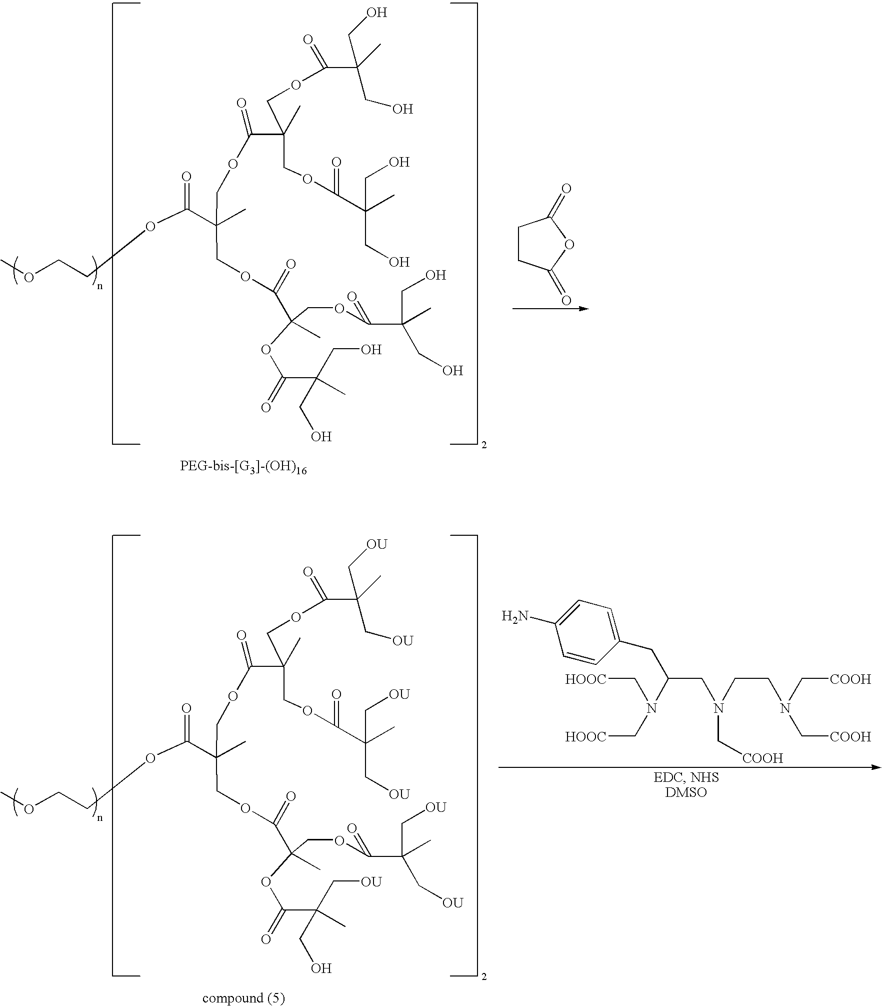

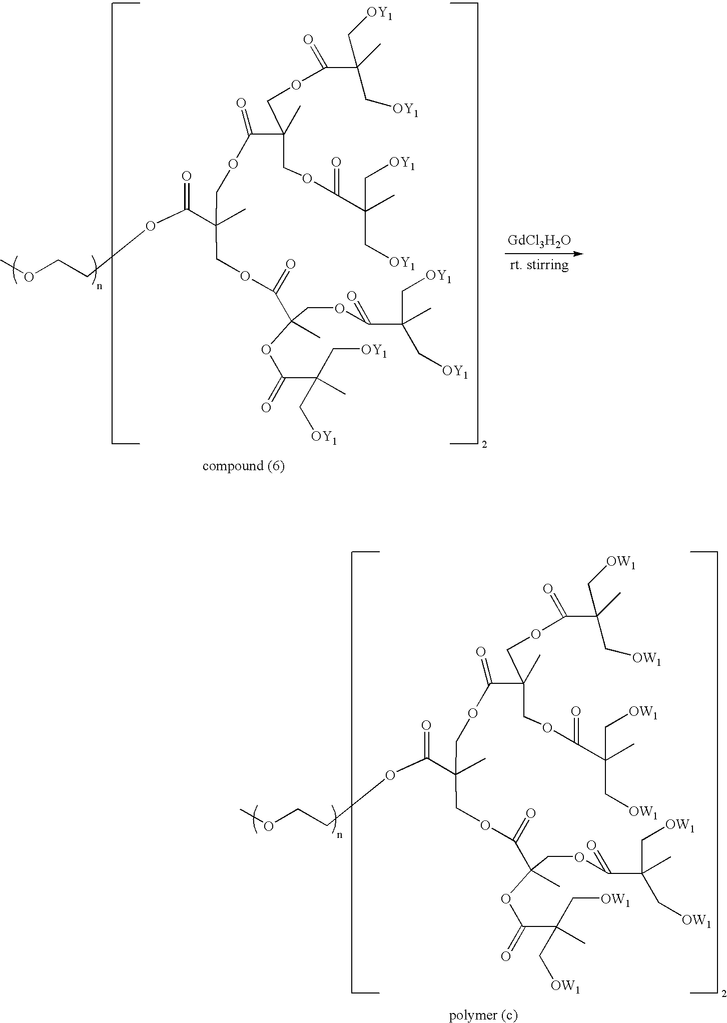

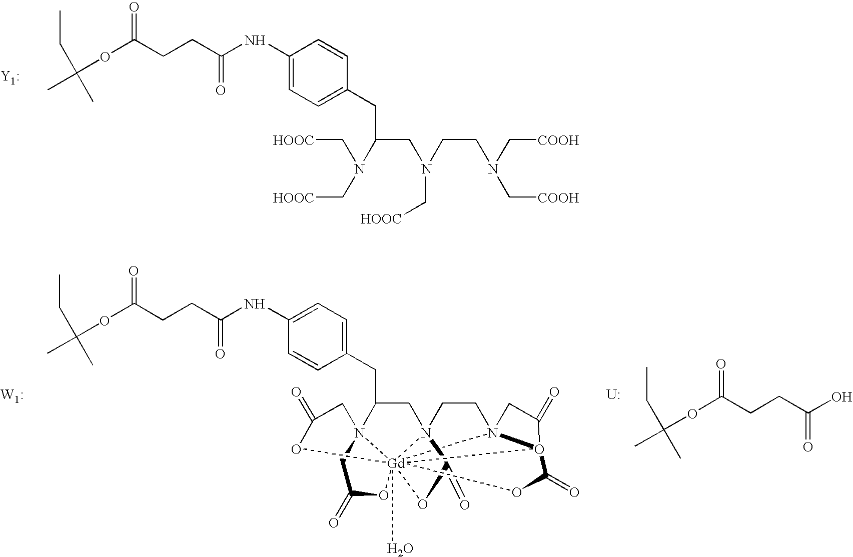

PEG-bis[G-3]-(OH)₁₆ (2.00 g, 0.35 mmol) and DMAP (0.17 g, 1.4 mmol) were dissolved in 75 mL of dichloromethane and succinic anhydride (0.63 g, 6.3 mmol) was added. The reaction mixture was stirred overnight and the mixture was precipitated in diethyl ether (2 L). The white precipitate separated was filtered and dried under vacuum, obtaining compound (5) (PEG-bis[G-2]-(OSA)₁₆. Physical measurement of the compound (5) is listed below:

¹H NMR (400 MHz, CDCl₃): δ 1.19 (m, 42), 2.57 (s, 64), 3.47 (t), 3.63 (bs), 3.80 (m), 4.21 (m).

By following the general procedure, from a mixture of compound (5) (0.2 g, 0.028 mmol), EDC (0.077 g, 0.49 mmmol), NHS (0.056 g, 0.49 mmol) and DMAP (0.059 g, 0.49 mmol) in DMSO (10 mL) with 2-(4-aminobenzyl)diethylenetriaminepentaacetic acid (0.242 g, 0.49 mmol) in 10 mL of DMSO, the compound (6) (PEG-bis[G-2]-(SA-NH-Bz-DTPA)16) with a yield 51 was synthesized. Physical measurement of the compound (6) is listed below:

IR (cm⁻¹): 3448, 2916, 1736, 1648, 1114. ¹H NMR (400 MHz, DMSO-d₆): δ 0.94, 1.12 (2s, 42), 2.38 (s, 64), 3.05 (t), 3.49 (bs), 4.13 (m), 7.10 (d), 7.57 (d).

The polymer (C) containing metal cation Gd³⁺ with rigid linker was prepared by adding a stoichiometric amount of GdCl₃.6H₂O into compound (6) in water. The solution was vigorously stirred for 4 hr at room temperature. The pH maintained between 6.0-6.5 using 1N NaOH solution. The progress of the reaction was followed by FTIR. The absence of free gadolinium ions were tested by using xylenol orange indicator at pH 5.8 (acetate buffer). The complexes were filtered using 0.45 μm filter and lyophilized.

T₁ Relaxation Measurements

The Gadolinium loaded dendritic polymer (A)˜(C) containing metal cation Gd3+ with rigid linker were evaluated for their capacity to alter the relaxation rate of water using a NMR spectrometer (20 MHz) with standard pulse program of inversion-recovery (IR). All the dendritic polymer (A)˜(C) (PEG-core dendrimers with rigid structure (Z is DTPA residual group with

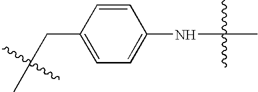

group) were analyzed using an initial concentration of 1 mmol in water and compared with PEG-core dendrimers as disclosed in U.S. Patent Publication No. 20070154390 without rigid structure (Z is DTPA residual group without

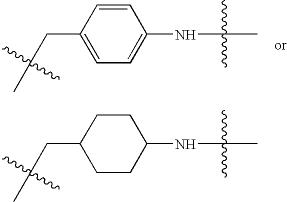

group). The results were shown in Table 1.

TABLE 1

| | No. of Gd/ | Relaxivity (mM · S)⁻¹ | | | |
|---|---|---|---|---|---|
| | dendrimer | Mol. | Mol. | Ionic | Ionic |
| Dendrimer | (ICP-AES) | r1 | r2 | r1 | r2 |
| Polymer (A) | 2.1 | 12.7 | 12.9 | 6.0 | 6.1 |
| Polymer (B) | 5.4 | 80.6 | 84.7 | 14.9 | 15.7 |
| Polymer (C) | 9.0 | 137.1 | 149.4 | 15.2 | 16.6 |
| Gd-PEG-G₁-(ODTPA)₄ | 3.3 | 15.8 | 17.9 | 4.8 | 5.4 |
| Gd-PEG-G₂-(ODTPA)₈ | 6.0 | 38.3 | 37.4 | 6.4 | 6.2 |
| Gd-PEG-G₃-(ODTPA)₁₆ | 11.7 | 75.7 | 77.5 | 6.5 | 6.6 |

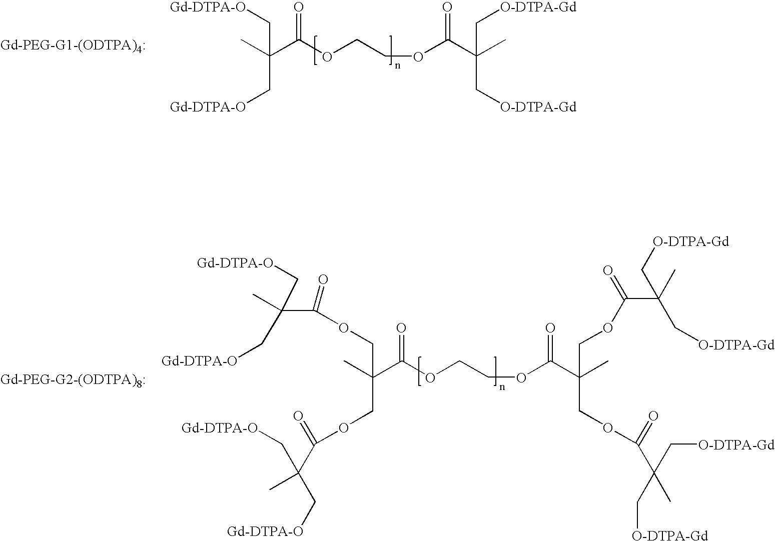

Gd-PEG-G3-(ODTPA)₁₆:

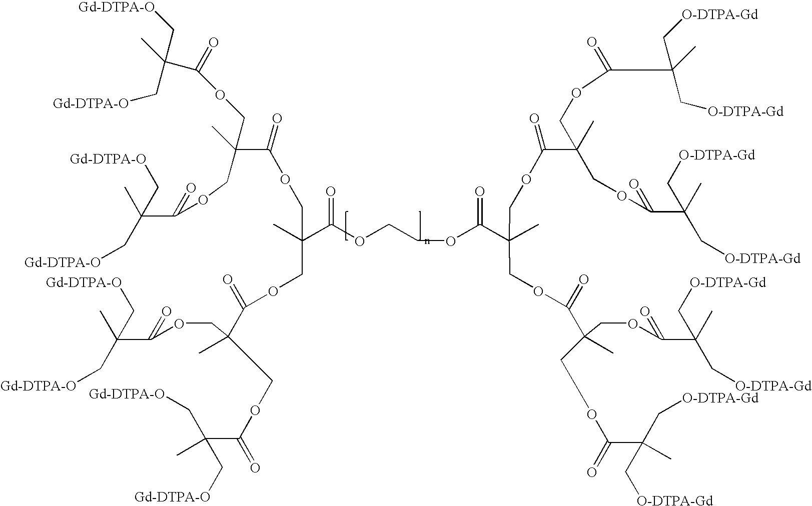

It should be noted that the dendritic polymers provided by the invention have an expected numbers of metal cations (for example, the expected numbers of Example 2 is 8 and the expected numbers of Example 3 is 16). However, due to the steric effect and uncertain factors from chemistry synthesis, the actual average numbers of metal cations are in general less than the expected numbers (for example, the actual average numbers of Example 2 is 6.0 and the actual average numbers of Example 3 is 11.7. Since dendritic polymers with different actual numbers of metal cations have various actual chemical structures, we use the chemical structure with the expected numbers of metal cations to generally represent all likely dendritic polymers with actual numbers of metal cations (which are prepared from the same synthetic process).

Accordingly, the water proton relaxation results indicate that polymers (A)˜(C) as disclosed in Examples 1-3 are having an inherent nature to act as contrast-enhancing agents. Generation 2 and 3 of dendritic polymers (B) and (C) showed higher relaxivity values than generation 1 of dendritic polymers (A). Further, the relaxivities of the dendritic polymers having rigid structure (DTPA residual group with

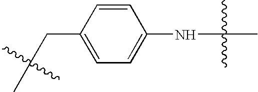

group) of the inventions) were found to be much higher than the dendritic polymers without rigid structure (Z is DTPA residual group) due to the more number of Gd³⁺ ion doping cites as well as more rigid nature of Bz-DTPA dendrimers.

One technical characteristic of the invention is to provide a multiple dendritic polymer carrier carrying a plurality of paramagnetic gadolinium ion or analyte-specific moieties, processing a unique magnifying ability with geometric series, so as to greatly increase signal strength of per unit nuclear molecular imaging contrast agents. Another technical characteristic of the invention is to introduce rigid linker (such as DTPA residual group with

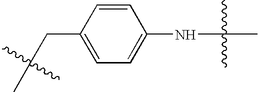

group) into dendritic polymer by chemical molecular design. As shown in Table. 1, the relaxivity of the magnetic resonance imaging contrast agent is proportional to the rigidity thereof. The magnetic resonance imaging contrast agents with rigid structure exhibits superior relaxivity, thereby avoiding imaging agent easily breaking through the skin cell in the blood and the drawback of easy metabolism by the human body.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A dendritic polymer, comprising structures as formula (I):
PDX-Z-L)ᵢ)ⱼ formula (I)
wherein P is CH₂CH₂O₁, 1 is not less than 1, and j is not less than 2;
D is independent and comprises a C₃₋₃₀ dendritic moiety having n oxygen residue, and n is not less than 3, and D respectively bonds with P and X by the oxygen residual groups;
X is a C₃₋₃₀ moiety having bi-functional groups;
Z is independent and comprises a C₃₋₂₀ moiety having a plurality of functional group, wherein the functional groups are selected from a group consisting of carbonyl, carboxyl, amine, ester, amide, or chelate group, and Z respectively bonds with X by a 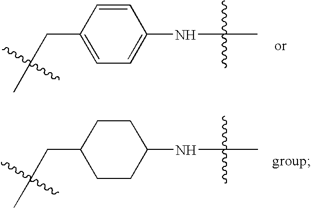
L is independent and a metal cation; and
i is more than 1.

2. The dendritic polymer as claimed in claim 1, wherein D comprises 2,2-dihydroxymethyl propanoic acid and residual groups of the derivative thereof.

3. The dendritic polymer as claimed in claim 1, wherein D is 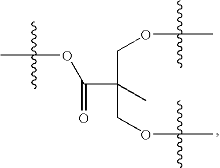
wherein i is 2.

4. The dendritic polymer as claimed in claim 1, wherein D is 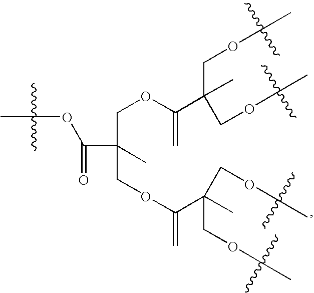
wherein i is 4.

5. The dendritic polymer as claimed in claim 1, wherein D is 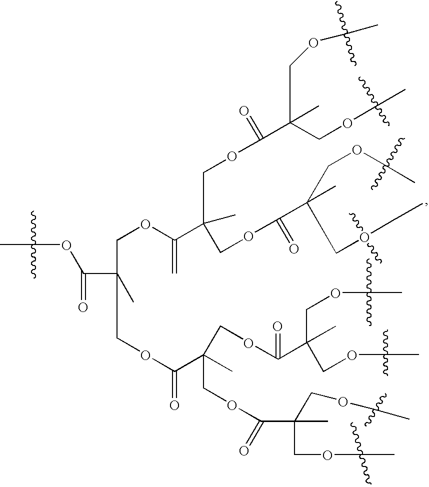
wherein i is 8.

6. The dendritic polymer as claimed in the claim 1, wherein L is Gd³⁺.

7. The dendritic polymer as claimed in the claim 1, wherein Z comprises a metal-chelated group.

8. The dendritic polymer as claimed in the claim 1, wherein Z comprises a residual group of ethylenedinitrilo tetraacetic acid (EDTA) or a residual group of ethylenediimino dibyric acid (EDBA) which with terminal 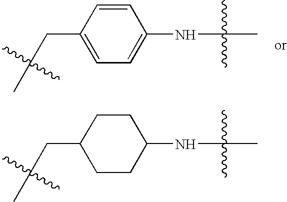
group.

9. The dendritic polymer as claimed in the claim 1, wherein Z is 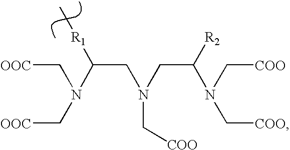
wherein R₁ is 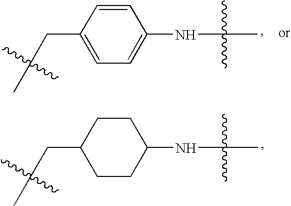
and R₂ is H, methyl, ethyl, or propyl group.

10. The dendritic polymer as claimed in the claim 1, wherein X is

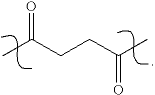

11. The dendritic polymer as claimed in the claim 1, wherein the dendritic polymer serves as a magnetic resonance imaging contrast agent.

12. A magnetic resonance imaging contrast agent, comprising the dendritic polymer as claimed in the claim 1.
